(19) United States
(12) Patent Application Publication
(10) Pub. No.: US 2006/0008823 A1
(43) Pub. Date: Jan. 12, 2006

(54) DNA profiling and SNP detection utilizing microarrays

(76) Inventors: Jennifer T. Kemp, Chapel Hill, NC (US); Shan X. Wang, Portola Valley, CA (US); Chris D. Webb, Scotts Valley, CA (US); Robert L. White, Stanford, CA (US)

Correspondence Address: LUMEN INTELLECTUAL PROPERTY SERVICES, INC., 2345 YALE STREET, 2ND FLOOR, PALO ALTO, CA, 94306, US

(21) Appl. No.: 11/125,558

(22) Filed: May 10, 2005

Related U.S. Application Data
(60) Provisional application No. 60/570,952, filed on May 12, 2004.

(51) Int. Cl. C12Q 1/68

(52) U.S. Cl. 435/6

(57) ABSTRACT

The present invention provides methods for rapidly identifying and distinguishing between different DNA sequences utilizing short tandem repeat (STR) analysis and DNA microarrays. Specifically, these methods facilitate the deduction of a target molecule's identity, length, and number of STRs. In an embodiment, a labeled STR target sequence is hybridized to a DNA microarray carrying complementary probes. These probes vary in length to cover the range of possible STRs. The labeled single-stranded regions of the DNA hybrids are selectively removed from the microarray surface utilizing a post-hybridization enzymatic digestion. The number of repeats in the unknown target is deduced based on the pattern of target DNA that remains hybridized to the microarray. The DNA profiling techniques described herein are useful for performing forensic analysis to uniquely identify individual humans or other species.

### CROSS-REFERENCE TO RELATED APPLICATIONS

This application claims priority from a provisional patent application No. 60/570,952, filed May 12, 2004, the entire content of which is incorporated herein by reference.

### STATEMENT REGARDING FEDERALLY SPONSORED RESEARCH OR DEVELOPMENT

The present invention was supported in part by grant number NOOO14-02-1-0807 from the U.S. Defense Advanced Research Projects Agency (DARPA). The U.S. Government has certain rights in the invention.

### BACKGROUND OF THE INVENTION

1. Field of the Invention

The present invention relates generally to DNA profiling and more particularly to STR profiling analysis utilizing DNA microarrays and to methods of deducing the identity and length of target molecules by way of enzymatic treatment of hybridized DNA. The present invention also relates to methods for improving sensitivity and accuracy of the STR profiling using magnetic detection for DNA microarrays, and to methods for extending the magnetic detection analysis to SNP analysis. All methods disclosed herein are useful for unique identification of individual humans or other species in forensic science.

2. Description of the Related Art

Especially in the field of forensic science, DNA-based techniques for the identification of individuals are becoming increasingly relied upon. Today, several techniques exist for forensic DNA profiling, which is also referred to as “DNA fingerprinting”. The method that the FBI and the British courts have accepted for use in forensic science is based on the tandem repeats present in the human genome. The variable number tandem repeats (VNTR) scheme is based on the long tandem repeat loci, those with up to about 100 repeats, each of about 8 to 80 base pairs in length. The short tandem repeat (STR) scheme is generally based on loci with about 3 to 15 repeats, each with between 3 and 5 base pairs.

The shorter repeats are more often used in forensic analysis, since the short repeat regions are readily amenable to PCR amplification. Longer repeat regions can be many thousands of bases in length and are more difficult to amplify. More importantly, since the final measurement is the number of repeats present at a given locus, it is more feasible to accurately measure repeat length for shorter regions. It is tractable to distinguish between four and five repeats, for instance, while it is more difficult to distinguish between fifty and fifty-one repeats. In the VNTR method, the measured repeat lengths are binned into fractions having a width of several repeats, therefore reducing the precision of the conclusion. Accurate determination of number of repeats is feasible with the shorter repeats.

In the noncoding regions of the genome, there are many loci where a particular sequence of DNA is repeated multiple times in direct succession. Some of these loci contain as many as 100 repeats. The number of tandem repeats at a given DNA locus varies between individuals.

The FBI and the forensic science community typically use 13 separate STR loci (the core CODIS loci) in routine forensic analysis. If two DNA samples have identical lengths at all 13 loci, the probability that the two samples originated from the same individual is approximately ten billion to one. The courts generally accept this identification as definitive evidence that the individuals in question are the same. CODIS refers to the Combined DNA Index System that was established by the FBI in 1998 based on 13 STR loci.

Generally, to perform a DNA profiling experiment based on STR analysis, the regions of DNA corresponding to each of the 13 STR loci are excised from the sample DNA using the appropriate restriction enzymes. The regions are then amplified using PCR and labeled with a dye or fluorescent molecule. The length of the DNA molecules is then determined using polyacrylamide gel electrophoresis (PAGE) or other known electrophoretic separation techniques, see, e.g., John M. Butler “Forensic DNA Typing” Academic Press, 2001.

Electrophoresis is a separation technique based on size, i.e., shorter DNA molecules migrate more rapidly down a gel or capillary than longer DNA molecules. The population of molecules (in this case, STR regions) is thus separated by size (or repeat length), and the final position of the DNA is determined by visualizing the staining pattern of the dye or fluorescent molecule. There exist miniature systems with an array of electrophoretic columns for this measurement. It is believed that STR analysis will remain the technique of choice in forensic science for DNA identification for the next decade, and that the number of loci used in this analysis will perhaps increase from 13 to 20.

Another known DNA profiling technique is single nucleotide polymorphism (SNP) analysis. In this method, a single region from the coding region of a gene from a known sample is compared with the analogous region from an unknown sample (for example, comparing a suspect's DNA sample with an unknown perpetrator's DNA sample collected from a crime scene). Currently, the region used is from chromosome 6. If the two regions are not identical in sequence, the suspect is eliminated as the perpetrator of the crime. However, if the sequences are identical, there is a 5% probability that the two samples came from the same individual. Since this probability is low, the identification value of the SNP approach is limited. In the case of a match, the analysis must proceed to the more definitive STR technique.

Several other variations of DNA analysis are used in forensic science. Another type of analysis involves mitochondrial DNA. Mitochondrial DNA is maternally inherited in a haploid manner, and can be used to determine familial relationships. Also, the X and Y chromosomes identify the sex of a subject. U.S. Pat. No. 4,396,713 issued to Simpson et al. discloses a method of restricting endonuclease digestion of the mitochondrial DNA to provide for substantial cleavage of kDNA network. The resulting electrophoretic profiles of the digest can be used for distinguishing organisms and specific strains. U.S. Pat. No. 6,251,592, issued to Tang et al., discloses some STR markers for DNA profiling. However, these STR markers are not in the CODIS.

The aforementioned DNA analyses are based on electrophoresis, a rather mature technology. Although still in their infancy, several DNA profiling methods using microarrays have been proposed.

R. Radtkey et al., in “Rapid, high fidelity analysis of simple sequence repeats on an electronically active DNA chip” Nucleic Acids Research, 28:E17 (2000), offer a high stringency approach for discriminating STR alleles based on active microarray hybridization. A sandwich hybrid is assembled, in which proper base stacking of juxtaposed terminal nucleotides results in a thermodynamically favored complex. The increased stability of this complex relative to non-stacked termini and/or base pair mismatches is used to determine the identification of STR alleles.

S. Stenirri et al., in “Single nucleotide polymorphism and mutation identification by microelectronic chip technology” Minerva Biotecnologica, 14:241-246 (2002), describe using microarray assays for identifying some common Italian mutations in the retina-specific ABC transporter gene, offering a specific example of SNP analysis.

These proposed DNA profiling methods require either a special electronically active DNA array to allow discrimination of subtle hybridization differences between repeats of similar lengths or sophisticated tiling probe sets to identify a single SNP. Unlike electrophoresis-based methods, none of these proposed methods has been widely adopted.

Other methods of using microarrays to specifically identify SNPs or VNTRs involve the use of ligase and/or polymerase. U.S. Pat. No. 6,150,095 discloses a technique in which the length of a VNTR is detected by hybridizing a target to a short probe to form a duplex, incubating the duplex with labeled nucleotides, and monitoring chain extension of the probe as an indication of the length of the variable number repeat section of the target. Other methods to determine the length of VNTR involve the use of ligation of tags combined with base extension. VNTR-based DNA profiling has largely been superseded by STR-based DNA profiling.

U.S. Pat. No. 5,753,439 discloses a method of using nuclease to nick mismatched base pairs followed by nick translation using DNA polymerase. With this method, target DNA is labeled and hybridized to a differently labeled probe. Mismatched bases due to differences in the length of the repeat region between the probe and the target are nicked with nuclease, and the remainder of the probe or target is elongated using nick translation, thereby displacing the label on the target or probe. This complicated method has not gained wide adoption.

There is a continuing need in the art for new and reproducible DNA profiling methods utilizing widely available microarrays for rapid determination of individual identify, which would be particularly useful in forensic science. The present invention addresses this need.

### SUMMARY OF THE INVENTION

In principle, a target containing an STR of unknown repeat length can be hybridized to an array displaying complementary probes that vary in length to cover the range of possible number of repeats. Differences in hybridization of target DNA to the various probes can then be used to determine the number of repeats. For example, a target with 10 repeats should bind more strongly to a probe with 10 repeats than to a probe with 5. However, in practice, the difference in hybridization efficiency of tandem repeats that are similar in length, e.g., 9 and 10 repeats, is very subtle and hard to detect.

The present invention provides new DNA profiling methods utilizing STR analysis and DNA microarray technology. According to an aspect of the invention, a variable length probe array (VLPA) is utilized to determine the length of an unknown STR with two novel techniques: a clamp sequence to ensure proper hybridization of the repeat sequences and a nuclease step to selectively remove single-stranded DNA sequences from the array.

In an embodiment, a post-hybridization enzymatic digestion of the DNA hybrids is employed to selectively remove labeled single-stranded regions of DNA and subsequently deduce the identity, length, and number of STRs of the target molecule. In addition to conventional fluorescent microarrays, the method could use high-sensitivity magnetic detector arrays such as spin valve arrays (SV arrays) and magnetic tunneling junction arrays (MTJ arrays) to perform magnetic detection of DNA labeled with magnetic substances. The method is further applied to SNP analysis combined with real-time denaturation of hybridized complexes followed by in situ detection using SV or MTJ arrays. These methods could be extended to detection of RNA and other chemical and biological species.

With the VLPA method, a biomolecule is identified by first hybridizing a labeled single-stranded target polynucleotide of length A to a single-stranded probe polynucleotide of length B and then selectively removing the label of the target polynucleotide when length A is greater than length B. In practice, Length A might be greater than, equal to, or less than length B.

In one aspect of the invention, the probe and target polynucleotides are deoxyribonucleic acid (DNA). This aspect can be applied to the field of DNA profiling, in which different DNA sequences are identified and distinguished in order to identify an individual. In this case, the probe and target polynucleotides include a finite number of short tandem repeat (STR) sequences. The lengths of the probe and target are determined by the number of STR sequences contained in the probe and target, respectively. The target polynucleotides are labeled at their 5′ or 3′ ends with a fluorescent dye, a superparamagnetic particle, or a synthetic antiferromagnetic particle.

In one embodiment, the fluorescent dye is Cy3 or Cy5. Targets can be end-labeled with a chemical means, biological means, or with a physical linker. Alternatively, the target and/or probe could be labeled internally.

Single-stranded DNA probes of varying length are attached by either the 5′ or 3′ end to the surface of a microarray in known, predetermined positions. Each position is a separate feature. The probes can be attached by modifying the probes with a chemical entity and by allowing the ends of the probes to attach, either covalently or noncovalently, to the microarray surface.

In some embodiments, the probes are modified with a sulfur-containing group, such as a thiol group, and the probes are attached to the substrate through a sulfur linkage. In some embodiments, the probes are modified with an amine group. Alternatively, a chemical or biological linker is used to attach the probes to the surface of the microarray.

The present invention also provides a fixed-length probe array (FLPA) method similar to the VLPA method. With the FLPA method, a biomolecule is identified by hybridizing a labeled single-stranded target of unknown length A to a single-stranded probe polynucleotide of predetermined fixed length B, detecting the number of polynucleotides that are hybridized to the probe, and determining length A based on this detection step. No post-hybridization enzymatic treatment is required.

It is therefore an object of this invention to detect STR sequences hybridized to DNA microarrays and to determine their length based on interpreting the results of selective removal of the single-stranded regions of DNA.

It is a further object of this invention to distinguish between STR sequences with various numbers of repeats using methods of detection such as fluorescence.

It is another object of this invention to improve sensitivity and accuracy of the STR analysis by incorporating a magnetic detection system for DNA hybridized to the surface of microarrays into the STR analysis.

It is a further object of this invention to uniquely identify individual humans or other species using the STR profiling and SNP analysis incorporated with microarrays.

Other objects and advantages of the present invention will become apparent to one skilled in the art upon reading and understanding the preferred embodiments described below with reference to the following drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates the steps of performing an STR analysis with the variable-length probe DNA profiling system according to an embodiment of the invention.

FIG. 2 illustrates an embodiment of the invention in which a clamp sequence is utilized to ensure proper hybridization of a target sequence to the probe sequence.

FIG. 3 illustrates the steps of performing an STR analysis with the fixed-length probe DNA profiling system according to an embodiment of the invention.

FIG. 4 exemplifies the steps of performing an SNP analysis using magnetic microarrays.

FIG. 5 presents two actual screen shots showing fluorescent images of a mircoarray (A) before and (B) after treatment with nuclease.

### DETAILED DESCRIPTION OF THE INVENTION

Nomenclature

Microarray a series of known DNA sequences attached in a regular pattern on a flat surface, such as a glass slide, and to which DNA molecules of unknown composition/sequence are hybridized for identification.

| | |
|---|---|
| STR | short tandem repeat. A short sequence of DNA that is found |
| | repeated sequentially at various loci in the human genome. |
| SNP | single nucleotide polymorphism. Any individual nucleotide |
| | which varies between individual humans. |
| Target | a DNA molecule of unknown sequence that is labeled and |
| | exposed to a microarray to allow hybridization to the probe. |
| Probe | a known DNA that is attached to a microarray and subsequently |
| | hybridized to the target. |
| Feature | an individual spot on a microarray. A feature represents one |
| | unique DNA sequence, although each feature contains multiple |
| | copies of that sequence. These features currently range in |
| | diameter from 20 to 100 microns. Feature size is anticipated to |
| | be smaller than 20 microns in future generations of microarrays. |
| Label | also called a “tag”. A molecule or particle that is attached to the |
| | biomolecule of interest and that is subsequently detected by the |
| | applicable detector system. A typical biomolecule-label scenario |
| | is a molecule of DNA which is covalently attached to a molecule |
| | of fluorescent dye such as Cy5. Within the context of the present |
| | invention, a label may also refer to a superparamagnetic nano- |
| | particle or a synthetic antiferromagnetic nanoparticle attached |
| | to a DNA molecule. |

Variable-Length Probe Array (VLPA) Method for STR Profiling

Detection of STR length using microarrays is hampered by the fact that the hybridization efficiency of repeats that are close in length is very similar. This makes it very hard to distinguish between STRs with similar numbers of repeats.

The present invention overcomes this problem with the VLPA method, utilizing targets and probes containing tandem repeats. Single-stranded DNA probes with varying number of repeats (and thus variable length) are end-attached to a microarray surface (each probe to a separate feature or “spot”).

Next, a sample containing fluorescently end-labeled single-stranded DNA with an unknown number of STRs is applied to the microarray and allowed to hybridize. After hybridization, the microarray is subjected to enzymatic digestion using a single-stranded endonuclease. This treatment removes single-stranded regions of DNA and consequently, removes the fluorescent label from the end of any single stranded region protruding from a hybridized duplex.

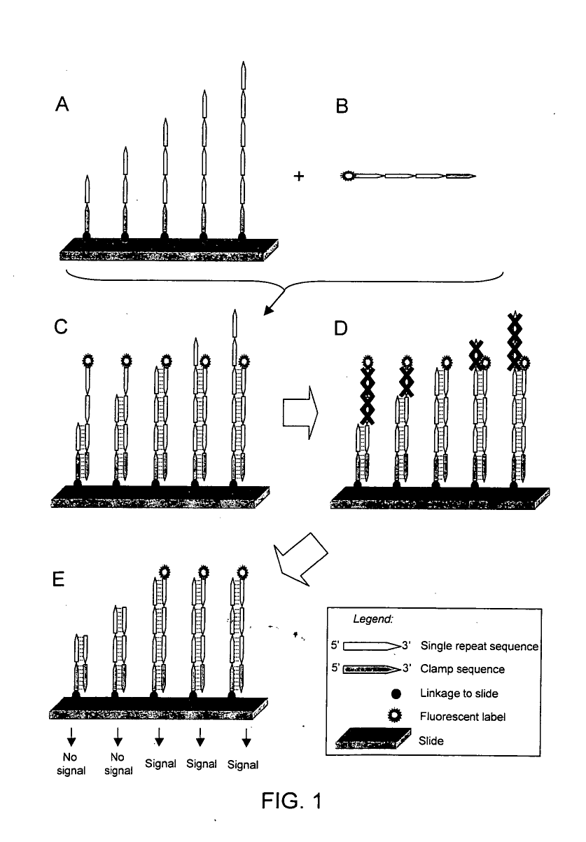

FIG. 1A illustrates an exemplary array with probes containing 1, 2, 3, 4, and 5 short tandem repeats. A target with three repeats is shown in FIG. 1B. Hybridization of this target to the array is depicted in FIG. 1C. Here, probe/target complexes that are formed after hybridization contain single-stranded regions when the probe and target are of different lengths. At this point, these single-stranded regions could optionally be stained with a dye or marker that specifically binds to single-stranded regions of polynucleotides.

After hybridization, the microarray is subjected to a process that selectively removes these single-stranded regions, either through chemical, biological, or physical means. In a preferred embodiment, the process used is enzymatic digestion using a single-stranded endonuclease (or exonuclease), which removes single-stranded regions of DNA but leaves double-stranded regions intact. Preferably, the endonuclease is S1 nuclease. In this scheme, the removal of single-stranded DNA also results in the removal of the detectable label on the target DNA, either the end-label or the single-stranded binding dye or marker, as illustrated in FIG. 1D, with X marks indicating digested regions of DNA.

The microarray is then assayed to determine which features have retained signal from the label after enzymatic treatment, either by fluorescence detection or magnetic detection, depending on the label used. Since the labeled end of the target DNA is removed when the target DNA is longer than the probe DNA attached to the microarray, only those features having a probe with length equal to or greater than that of the target DNA will retain signal after enzymatic treatment, as illustrated in FIG. 1E.

As describe above, the length of the unknown target DNA is deduced from the results of the enzymatic digestion of the hybridized microarray and is determined to be equal to the length of the shortest probe that yields signal after enzymatic digestion. In this detection scheme, three possible outcomes exist for the target-probe hybridization pattern.

The first possible outcome is that the labeled target may have more repeats than the probe attached to the microarray. As described below, we use a clamp sequence to ensure that the target DNA anneals to the probe so that the single stranded region of the target DNA will protrude from the hybridized complex into solution (see, e.g., probes with 1 and 2 repeats in FIG. 1C). When the microarray is treated with single-stranded endonuclease, the single-stranded region of target DNA and the fluorescent label are removed (see, e.g., probes with 1 and 2 repeats in FIG. 1D and FIG. 1E), resulting in a loss of signal detected from this feature.

The second possible outcome is that the target and the probe may have an equal number of repeats, in which case no single-stranded DNA is present (see, e.g., probe with 3 repeats in FIG. 1C). In this case, the endonuclease treatment has no effect on the hybridized complex and the fluorescent moiety is not removed (see, e.g., probe with 3 repeats in FIG. 1D and FIG. 1E). The signal detected from this feature remains unchanged.

The third outcome occurs if the target has fewer repeats than the probe, in which case a region of single-stranded probe DNA protrudes from the hybridized complex (see, e.g., probes with 4 and 5 repeats in FIG. 1C). Although this single-stranded region of probe DNA is removed during the endonuclease treatment, the target DNA is not digested and the fluorescent label remains attached. Thus, the signal detected from this feature remains unchanged after endonuclease treatment.

Thus, following endonuclease treatment, the fluorescent signal will only remain on features containing probes with an equal or greater number of repeats than the target, as illustrated in FIG. 1E. The fluorescent signal can now be read using a standard microarray scanner without any additional special equipment. For any given STR sequence in an unknown sample, the number of repeats is determined to be equal to the number of repeats in the shortest probe that yields signal after hybridization and enzymatic treatment.

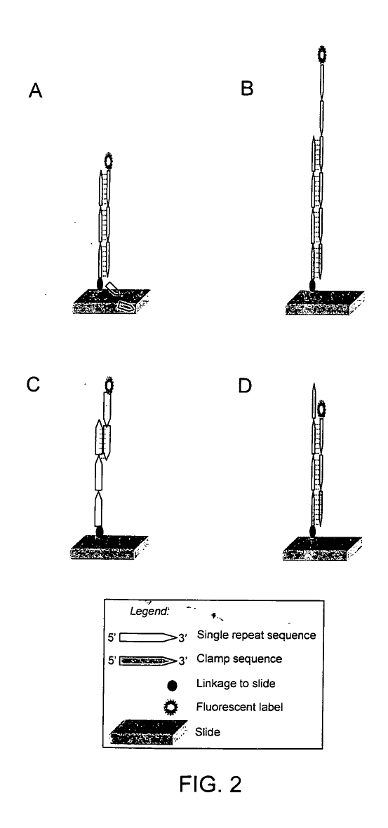

A key requirement is that the target anneals to the probe in the proper register. That is, it must anneal without misaligned repeats or “slippage”. For example, in FIG. 2A, a target with more repeats than the probe could anneal such that the fluorophore would not be removed by nuclease treatment and an improper signal would be retained.

Conversely, in FIG. 2C, a target with fewer repeats than the probe could anneal such that the fluorophore would be removed by the nuclease, and a signal would be improperly lost. Thus, the VLPA method requires that the 3′-most repeat of the target DNA anneals to the 5′-most repeat on the array probe (in a system where the probe is 5′ end attached to the array).

To ensure that the target anneals to the probe in the proper register, a “clamp” sequence could be added to both the target and probe DNA. The clamp sequence is added at the microarray-proximal end of the probe, and its complement is added at the label-distal end of the target (see, FIG. 2B and FIG. 2D).

The clamp sequence can be more GC-rich than the repeat sequences, thereby biasing the hybridization to the proper register. Using a clamp sequence greatly simplifies the analysis of the variable-length probe profiling method. While this method is possible without a clamp sequence, the addition of this clamp sequence to the method ensures that an obvious and measurable signal difference will be generated between positive and negative probes without having to resort to cumbersome and specialized hybridization conditions.

In some embodiments, spacer sequences are utilized as a further refinement to the VLPA method. For example, the probe polynucleotide could contain a spacer that would allow the repeat sequence to protrude into solution and away from the surface of the microarray. A spacer sequence could also be inserted in the target polynucleotide, between the repeats and the end-label. The presence of the space sequence could enhance the robustness of the assay by reducing interference between the end-label and the nuclease.

Fixed-Length Probe Array (FLPA) Method for STR Profiling

The FLPA method is a variation of the VLPA method described above. Probes with fixed length are employed to deduce the length and number of repeats in a given STR sequence. Although the FLPA method also utilizes the microarray technology, enzymatic treatment of the microarray is not required. The experimental procedure is otherwise similar to the VLPA method described above.

With the FLPA approach, fixed-length probes attached to the microarray are designed with a length greater than the longest DNA molecule expected to be detected in an unknown target sample that is to be hybridized to the chip. When the target is shorter than the probe and is present in multiple copies, it can hybridize to the longer probe multiple times, depending on its length relative to the length of the probe. Therefore, a shorter target molecule (with fewer repeats), will hybridize in more places along the length of the probe than a longer target molecule. A probe with fewer (longer) target molecules hybridized will yield a smaller signal on the microarray than will a probe with a larger number of shorter target molecules, assuming that the number of molecules in the target sample is in excess to the number of molecules displayed on the microarray.

When the probe and target polynucleotides are DNA, the probe and target include a finite number of STRs, and the length of the probe and target are determined by the number of STRs in the probe and target, respectively, this method is ideally suited to DNA profiling. For DNA profiling, the probe should contain at least twice the number of STRs as the target.

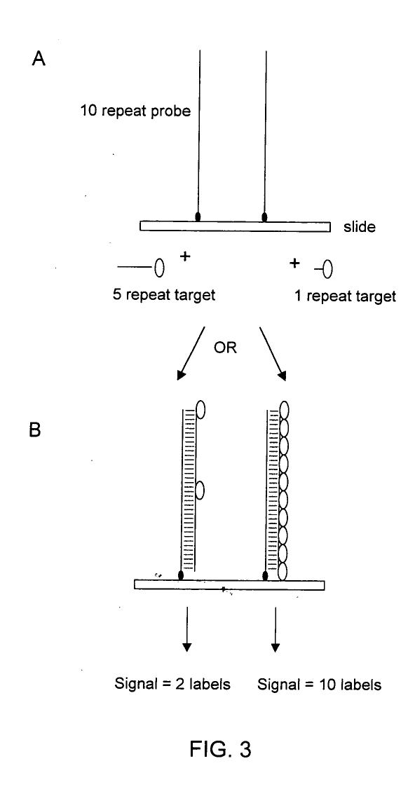

For example, consider a 100 base pair end-labeled probe with 10 repeats, each with 10 base pairs, as shown in FIG. 3A. If the target is 50 base pairs in length (5 repeats), two separate end-labeled target molecules could hybridize to a single probe molecule, as shown in FIG. 3B. If the target is only 10 base pairs long (1 repeat), ten separate molecules could hybridize to a single probe.

A sensitive detection system such as the spin valve system or the MTJ system is quantitative enough to discriminate between one label versus two or many. Thus, the number of molecules that anneal onto a fixed-length probe can be readily measured and the length of the STR can be deduced from this information, since the length of the STR in the probe is known. This method is most accurate when the surface concentration at the hybridization sites of the probe is smaller than that of the target such that the probability of multiple targets annealing to a fixed length probe with complementary tandem is very high.

Since detection of a single hybridization event is possible using magnetic detection, accurate detection will be feasible by printing microarrays with very low concentrations, e.g., down to tens or even a few number, of probe DNA on each feature, even with very small quantities of target DNA in the unknown sample.

Choice of Label and Detection System

Both the VLPA and FLPA techniques described above may be carried out with any detection system, for instance, a standard fluorescence technology. In the experiments disclosed herein, the probe DNA was attached to a standard microarray. The DNA was end-labeled at the 5′ end with a fluorophore that emits light when excited under the appropriate wavelength. The signal from the fluorophore is detected using a standard fluorescent scanner.

The sensitivity and accuracy of the VLPA method and especially the FLPA method would be improved with the state-of-the-art detection systems that are quantitative and capable of single-label detection. For applications requiring a high level of accuracy, such as distinguishing between a sequence with 50 repeats and one with 51 repeats, detection systems using either superparamagnetic or synthetic antiferromagnetic nanoparticles to label the target DNA are preferred.

A suitable candidate is a biomagnetic gene chip (MagArray™) developed by Stanford University. This technology uses spin valves or magnetic tunneling junction (MTJ) detectors to detect paramagnetic nanoparticles. The magnetic nanoparticles are used instead of a fluorophore to label the DNA. This system is capable of single-nanoparticle detection. Therefore, the magnetic detection system can detect a single hybridization event on a microarray, allowing single-label detection and accurate quantitation of the number of labels detected from a single feature over a range of about three orders of magnitude. Additionally, the magnetic detection system is quantitative and can distinguish between features having one, ten, one hundred nanoparticle-labeled DNA molecules hybridized and beyond.

SNP Analysis Using Microarrays and Real-Time Hybridization Detection

SNP detection using fluorescent microarrays is not yet optimized with fluorescence-based DNA microarray technology since hybridization detection is not sensitive enough to readily detect single base pair differences. However, the magnetic detection system for microarrays can be applied to SNP analysis. Using magnetic detection, the temperature can be raised during detection of hybridization, causing single-base mismatched molecules to denature before perfectly matched molecules. Hybridization is temperature-dependent; the annealing of two complementary molecules of single-stranded DNA occurs at or below a temperature which is determined by the length, nucleotide content, and percent of complementary nucleotides of the two molecules. Molecules which have a larger number of complementary bases anneal to form hybrids at higher temperatures than those with smaller numbers of complementary bases. Additionally, denaturation of complementary molecules occurs at higher temperatures with strands that have a larger number of complementary bases.

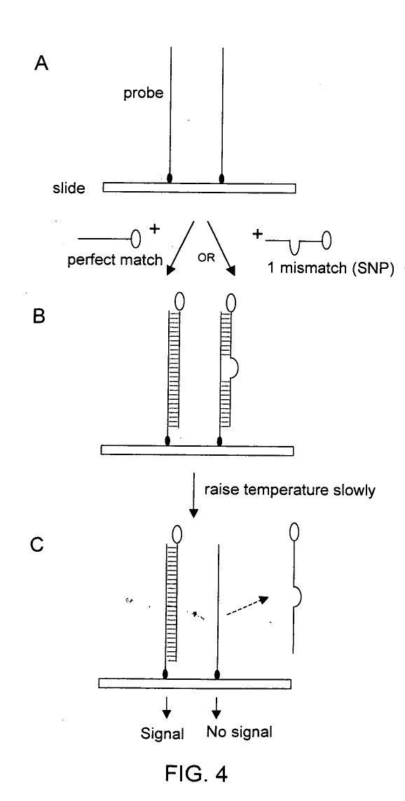

As illustrated in FIG. 4, this feature can be utilized for SNP detection using microarrays. By gradually raising the temperature of the apparatus to which DNA hybrids are attached, hybrids are denatured in an order that depends on their melting temperature (FIG. 4C). For example, a 20 base pair hybrid with one mismatch denatures at a lower temperature than does a 20 base pair hybrid with no mismatches. By examining which features of the microarray exhibit decreased signal upon raising temperature, features that contain hybrids with SNPs can be identified in real-time. Thus, another embodiment of this invention discloses a method of detecting single nucleotide polymorphisms comprising attaching at least one polynucleotide probe the surface of a microarray (FIG. 4A), hybridizing at least one labeled single-stranded polynucleotide target to the probe to form probe/target hybrids (FIG. 4B), denaturing the hybrids, and monitoring the denaturation in real time as labeled targets are removed from the microarray (FIG. 4C). The probe/target hybrids are preferably denatured with heat, but they could also be denatured with chemicals, such as salt solution.

It is estimated that there are 300,000 SNPs in the human genome. Mathematically, only about 20 sequence variations are necessary for unique identification of an individual. However, the exact SNP identifiers are yet to be determined by a consortium of scientists collecting the information about the positions and identities of SNPs (see, e.g., “The rough guide to the genome,” Nature, 425:758-759 (2003)).

This temperature raising scheme can also be applied to the FLPA STR profiling system described above. Instead of denaturing SNPs, the real-time temperature increase can be used to denature shorter hybrids (with fewer repeats) at lower temperatures than longer hybrids (with more repeats).

Experiments

Identical hybridizations were independently performed on three identical microarrays. The first microarray was processed and analyzed immediately after hybridization. This microarray served as a pre-nuclease incubation control (Control 1). The second was subjected to a post-hybridization incubation in S1 nuclease buffer without S1 nuclease and served as a control for the nuclease incubation (Control 1). The third microarray was subjected to a post-hybridization incubation in S1 nuclease buffer containing S1 nuclease (Nuclease incubation). The third microarray was otherwise treated identically to the second microarray in terms of duration and temperature of incubation. The third microarray thus served as the test sample.

These microarrays were prepared using CodeLink® activated slides, available from Amersham of Piscataway, N.J., and 5′ amine-modified oligonucleotide probes, available from Qiagen of Alameda, Calif. The oligonucleotides (5′-3′) comprise a 5′ amine group to facilitate attachment to the microarray, a C6 spacer, a 15 base pairs (bp) clamp sequence (not underlined), and 1, 2, or 3 tandem repeats of a 10 bp sequence ACGTGACTCT (underlined), as shown in Table 1 below.

Probes were printed onto microarrays from a solution containing the oligonucleotide at a concentration of 10 μM using an OmniGrid® microarrayer, available from GeneMachines of Ann Arbor, Mich. The post-printing processing of the microarrays was performed as recommended by the slide manufacturer.

TABLE 1

| | | | |
|---|---|---|---|
| Oligonucleotide | Function | Repeats | Sequence |

| | | | |
|---|---|---|---|
| JTK026-r | probe | 1 | [AminoC6] GTACCGGAATTCCGG ACGTGACTCT |
| JTK027-r | probe | 2 | [AminoC6] GTACCGGAATTCCGG ACGTGACTCT ACGTGACTCT |
| JTK028-r | probe | 3 | [AminoC6] GTACCGGAATTCCGG ACGTGACTCT ACGTGACTCT ACGTGACTCT |
| JTK028 | target | 3 | [Cy5] AGAGTCACGT AGAGTCACGT AGAGTCACGT CCGGAATTCCGGTAC |

Hybridization was performed using a target oligonucleotide, available from Qiagen of Alameda, Calif. The target comprises a Cy5 fluorophore on the 5′ end, three tandem repeats of a 10 bp sequence AGAGTCACGT (underlined) that was complementary to repeats on the probe, and a 15 bp clamp sequence (not underlined) that was complementary to the clamp on the probe, as shown in Table 1 above.

The target oligonucleotide was applied to the microarray at a concentration of 1 μM and the hybridizations were performed at 50° C. for 4-12 hours. After hybridization, the microarrays were washed 3 times in SSC buffer, according to the Amersham protocol, at room temperature and then submerged into buffer that was pre-equilibrated to 37° C. and that contained S1 endonuclease (Invitrogen, Carlsbad, Calif.) at 0.3 μl/ml in 1× reaction buffer.

Microarrays were then incubated in S1 endonuclease solution at 37° C. for ten minutes with intermittent agitation. After nuclease digestion, microarrays were washed three times in buffer containing 0.01×SSC and 0.01% SDS, three times in buffer containing 0.01×SSC, and dried. Microarrays were assayed for fluorescent signal at 635 nm using a GenePix 4000® fluorescent scanner (Axon Instruments, Foster City, Calif.) set to scan at 400 PMT.

The experiments were performed using a 10 minute S1 nuclease incubation, which was determined to be optimal. In other experiments (data not shown), some digestion was apparent after as little as 2 minutes, while loss of signal due to overdigestion was observed when incubation proceeded 15-30 minutes or longer. The signal differential between probes was greatest at 10 minutes.

Table 2 below shows the mean fluorescence intensities (expressed as a percentage of the 3-repeat probe intensity) plus or minus the standard error of the mean (SEM) calculated for each fluorescent dataset. We used GenePix® Pro software to determine the total fluorescent signal from each feature. Four separate arrays were analyzed for each treatment and the results were compiled as follows. For each oligonucleotide under each condition, data was collected from at least 6 separate features from the control experiments (hybridization experiment and buffer incubation), and from 14 separate features from each nuclease incubation experiment. Unpaired t-tests were used to calculate p values for the data from the nuclease treatment. In all experiments, background fluorescence was less than 5%.

TABLE 2
Control 1: After hybridization, no nuclease incubation

| | 3-repeat probe | 2-repeat probe | 1-repeat probe |
|---|---|---|---|
| A | 100 ± 10 | 104 ± 27 | 103 ± 11 |
| B | 100 ± 14 | 123 ± 13 | 101 ± 8 |
| C | 100 ± 5 | 121 ± 7 | 81 ± 4 |
| D | 100 ± 3 | 103 ± 4 | 89 ± 3 |
| Mean | 100 | 113 | 94 |

Control 2: Incubation in nuclease buffer without nuclease

| | 3-repeat probe | 2-repeat probe | 1-repeat probe |
|---|---|---|---|
| A | 100 ± 11 | 117 ± 26 | 120 ± 12 |
| B | 100 ± 11 | 147 ± 12 | 101 ± 6 |
| C | 100 ± 9 | 137 ± 15 | 97 ± 9 |
| D | 100 ± 3 | 127 ± 5 | 97 ± 5 |
| Mean | 100 | 132 | 104 |

Nuclease incubation

| | 3-repeat probe | 2-repeat probe | 1-repeat probe |
|---|---|---|---|
| A | 100 ± 5 | 42 ± 2 | 7 ± 0.3 |
| B | 100 ± 8 | 71 ± 5 | 32 ± 1 |
| C | 100 ± 4 | 59 ± 3 | 22 ± 1 |
| D | 100 ± 6 | 77 ± 6 | 19 ± 1 |
| Mean | 100 | 62 | 20 |

The fluorescence intensities for the control hybridization were similar between oligos with 1, 2, or 3 repeats. Likewise, the fluorescence intensities of the features incubated in buffer without S1 nuclease were similar for 1, 2, or 3 repeats. However, the fluorescent signal from the features with 1-repeat probes was substantially weaker than the signal from the features with 3-repeat probes on the microarray that was incubated in S1 nuclease. The features with 2-repeat probes showed a moderate decrease in signal relative to the 3-repeat probe. To quantitate the effects of the nuclease digestion on signals from the different probes, we analyzed four representative experiments that were performed identically but independently and calculated the mean fluorescence intensity from each probe as described in Materials and Methods.

On the two control arrays, the signal from the 1- and 2- repeat probes was not substantially reduced. In contrast, after S1 nuclease digestion, the signal from the 1-repeat probe was reduced approximately 5-fold compared to the signal from the 3-repeat probe (p<0.0001), and the signal from the 2-repeat probe was reduced by about 38% (p<0.0001). In other experiments, decreases in signal of as much as 20-fold have been observed from the 1-repeat probe (data not shown). No hybridization was observed of the target to a heterologous probe sequence (data not shown).

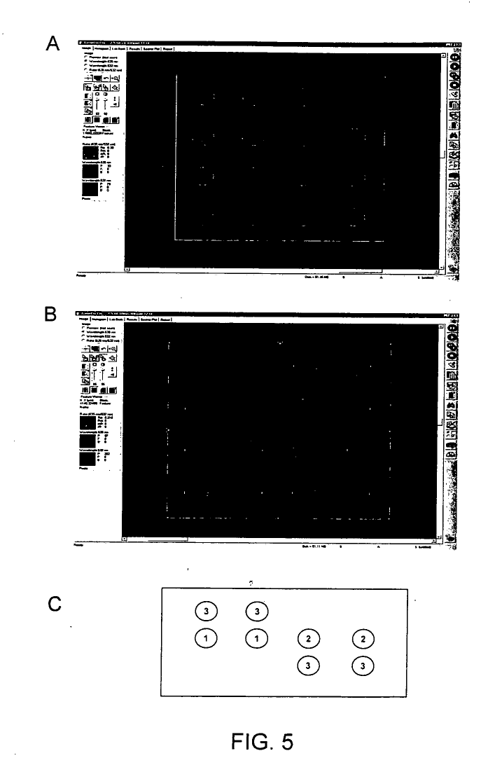

FIG. 5 shows fluorescent images of portions of several representative microarrays from the experiment. FIG. 5A shows the array after hybridization (Control 1). FIG. 5B shows the hybridized array after treatment with S1 nuclease for ten minutes at 37 degrees C. (Nuclease incubation). FIG. 5C is a map of the array with the number of repeats per probe shown in each circle. The microarray that was incubated in S1 nuclease buffer without S1 nuclease (Control 2) was similar in relative signal levels to the pre-nuclease control microarray (Control 1).

Compared to the pre-nuclease signals, all three of the probes have reduced signal. Several factors may explain this phenomenon. First, the overall decrease in signal may result from nonspecific activity of S1 nuclease against double-stranded DNA. The decrease may also simply be an experimental variation between different microarrays. Further testing and optimization of enzyme incubation protocol will determine the reason for the nonspecific post-nuclease decrease in signal.

The difference between the signals from the 2- and 3-repeat probes is smaller than the difference between signals from the 1- and 3-repeat probes. This may be due to steric hindrance of the enzyme by the label. That is, the 10-base single-stranded region that results from the hybridization of the 2-repeat probe and the 3-repeat target is not accessible to the nuclease because it is physically blocked by the large fluorescent molecule on the 5′ end of the target. Further testing and the insertion of a spacer sequence between the repeats and the label of the target may resolve this issue.

The experiments indicate that the S1 nuclease treatment results in reduced signal from features with fewer repeats than the target. These data are consistent with the expected pattern of nuclease digestion and supports the feasibility of the variable-length probe STR profiling method. To our knowledge, this work represents the first selective digestion of end labels of single-stranded DNA hybridized to probes of varying lengths attached to the surface of a microarray.

Application, Portability and Performance

The above experiments were directed to determining the length (and therefore number of repeats) of a single STR sequence. As one skilled in the art will appreciate, the methods described herein can be expanded to identify many different STR sequences on a single microarray in one experiment, which has practical applications in human profiling and identification.

Typical identification of a human being involves using 13 different STRs, each with 3-15 tandem repeats, in a profiling experiment. For each STR, a range of different lengths of probes must be represented as features on the microarray. Thus, as few as several hundred different features could be sufficient to uniquely identify an individual. For example, if 20 different features are required for identification of a single STR, only 260 features would be required to identify a human being. This number falls well within the range of features that can be represented on a single microarray.

Because current microarray technology allows hundred of thousands of unique features on a single chip, multiple copies of each feature can be incorporated into the assay to ensure accuracy. Using a single microarray, thousands of identical features can be compared to each other to distinguish between datasets with slightly different average fluorescence levels. The microarray design can also incorporate a variety of controls of similar length and sequence to the relevant sequences to eliminate background signal and ensure accuracy in relating the fluorescence levels to repeat number.

In practice, several complicating issues may arise with forensic specimens. Many STR alleles contain a partial repeat or other variation of an adjacent set of exact tandem repeats. Other situations requiring special consideration are heterozygosity, mixtures, or any other case in which two or more target sequences are present in an unknown sample. In such cases, additional probe sequences would be added to the microarray to cover each example of a possible known variant, and cross hybridization issues would be avoided by using precise control of hybridization conditions. The addition of a microfluidics system to the VLPA method could allow us to vary experimental conditions such as temperature or buffer and to make comparisons between hybridizations under several different conditions within a single experiment.

According to an aspect of the invention, a method of identifying an individual comprising the steps of obtaining a sample from the individual, isolating target polynucleotides from the sample, and determining the number of STR sequences present in the target polynucleotides using the methods described above. The DNA samples are obtained by conventional means well known to one skilled in the art. The target polynucleotides would be isolated by conventional means such that they contain at least one STR locus. Preferably, a variety of polynucleotides would be isolated, with each type of polynucleotide containing a different STR locus. STR loci can be found, for example, in the FBI's CODIS.

A STR/SNP detection (DNA profiling) system implementing the methods described herein can be fabricated with technologies similar to the very large scale integration (VLSI) technology. The spin valve and MTJ detectors themselves can be made in sub-micron size. Thousands to millions of detectors can therefore be integrated on a single microarray to result in a chip that is only several square centimeters in size.

In some embodiments, the DNA profiling system is integrated with a microfluidics system for sample preparation, hybridization, enzymatic digestion, and the like. In some embodiments, it is also integrated with an electronic system for detection readout. In some embodiments, the entire system is packaged to the size of a laptop computer or handheld device. This allows the profiling device to be carried into the field for use in forensic and military applications. Thus, another embodiment of this invention includes a device or apparatus implementing the above methods. Such a device comprises an array of polynuclotide probes of varying lengths attached to a solid substrate, a microfluidics system, a sensor or detection system for detecting label, and an electronic system for providing the detection result. In the case of STR profiling, the apparatus would have polynucleotide probes that are complimentary to at least one STR locus, such as those defined in CODIS.

The microarray-based profiling system of the present invention allows for rapid identification of DNA samples and other chemical and biological species. Particularly in the case of the magnetic detection system, the entire experiment could be performed in less than one hour. The sensitivity of the magnetic microarray eliminates the need for PCR amplification of the sample and greatly reduce the time required for sample preparation. The electronic readout from the magnetic microarray with tens of thousands of sensors takes only a few minutes due to the rapid sampling of spin valve or MTJ sensors.

The VLPA method described herein incorporates a nuclease treatment and specialized clamp sequences to allow robust and rapid STR length determination. The use of the clamp sequence to prevent slippage and ensure proper hybridization is a key innovation of the VLPA method. The sequences that flank the STRs in the human genome are the logical choice for these clamp sequences in practice. The insertion of a spacer sequence between the repeats and the fluorophore of the target oligonucleotide could also be a useful addition to enhance the robustness of the assay.

Although the present invention and its advantages have been described in detail, it should be understood that the present invention is not limited by what is shown or described herein. As one of ordinary skill in the art will appreciate, the DNA profiling methods disclosed herein could vary or otherwise modified without departing from the principles of the present invention. Accordingly, the scope of the present invention should be determined by the following claims and their legal equivalents.

We claim:

1. A method of identifying a biomolecule, comprising
hybridizing a labeled single-stranded target polynucleotide of length A to a single-stranded probe polynucleotide of length B; wherein
said length A is greater, equal to, or less than said length B; and
selectively removing said label of said target polynucleotide if said length A is greater than said length B.

2. The method of claim 1, wherein
said probe polynucleotide and said target polynucleotide are deoxyribonucleic acid (DNA).

3. The method of claim 1, further comprising
attaching said probe polynucleotide to a predetermined position on surface of a microarray.

4. The method of claim 3, further comprising
modifying said probe polynucleotide on its 5′ or 3′ end with a chemical entity to allow said end to attach covalently or noncovalently to said microarray surface.

5. The method of claim 3, further comprising
utilizing a chemical or biological linker to attach said probe polynucleotide to said microarray.

6. The method of claim 3, wherein
said probe polynucleotide contains a spacer sequence; and wherein
said spacer sequence allows a repeat sequence to protrude into a solution and away from said surface of said microarray.

7. The method of claim 1, wherein
said target polynucleotide contains a spacer sequence.

8. The method of claim 1, wherein
said probe polynucleotide and said target polynucleotide respectively includes a finite number of short tandem repeat (STR) sequences; and wherein
said length A and said length B are respectively determined by said number of STR sequences contained in said probe polynucleotide and said target polynucleotide, respectively.

9. The method of claim 1, further comprising
modifying said probe polynucleotide with a sulfur-containing group; and
attaching said modified probe polynucleotide through a sulfur linkage to a substrate.

10. The method of claim 1, further comprising
modifying said probe polynucleotide on its 5′ or 3′ end with an amine group.

11. The method of claim 1, wherein
after said hybridizing step, said probe polynucleotide and said target polynucleotide form a double-stranded probe/target complex; and wherein
differences in said length A and said length B result in single-stranded regions of said probe/target complex.

12. The method of claim 11, further comprising
staining said single stranded regions with a single-stranded binding dye or marker.

13. The method of claim 11, further comprising
removing said single-stranded regions.

14. The method of claim 11, further comprising
removing said single-stranded regions utilizing a chemical means, a biological means, a physical means, endonuclease digestion, S1 nuclease digestion, or exonuclease digestion.

15. The method of claim 1, further comprising
labeling said target polynucleotide on its 5′ or 3′ end with a fluorescent dye, a superparamagnetic particle, or a synthetic antiferromagnetic particle.

16. The method of claim 15, wherein
said fluorescent dye is Cy3 or Cy5.

17. The method of claim 1, further comprising
attaching said target polynucleotide to an end-label with a chemical means, a biological means, or a physical linker.

18. The method of claim 1, further comprising
labeling said probe polynucleotide, said target polynucleotide, or both, with at least one molecule at a position that is neither 5′ end nor 3′ end.

19. The method of claim 1, wherein
said probe polynucleotide, said target polynucleotide, or both contain a clamp sequence flanking repeats sequences thereof.

20. The method of claim 1, wherein
said probe polynucleotide, said target polynucleotide, or both, contain flanking sequences of random lengths on either side of repeat sequences thereof.

21. The method of claim 1, further comprising
detecting presence of said target polynucleotide hybridized to said probe polynucleotide.

22. The method of claim 1, further comprising
detecting presence of said target polynucleotide hybridized to said probe polynucleotide by fluorescence detection or magnetic detection.

23. A method of identifying an individual comprising
obtaining a biological sample from said individual;
isolating target polynucleotides from said sample;
labeling said target polynucleotides from said sample; and
determining, according to the method steps of claim 1, a number of short tandem repeat (STR) sequences present in said target polynucleotides.

24. The method of claim 23, wherein said target polynucleotides are complementary to at least one STR locus identified in a combined DNA index system.

25. An apparatus for implementing the method according to claim 1, comprising
an array of polynucleotide probes of varying lengths attached to a solid substrate;
a microfluidics system;
a sensor for detecting said label; and
an electronic system for providing a detection result.

26. The apparatus of claim 25, wherein
said polynucleotide probes are complementary to at least one STR locus.

27. The apparatus of claim 25, wherein
said sensor is capable of fluorescence detection, magnetic detection, or both.

28. A method for identifying a biomolecule, comprising
hybridizing a labeled single-stranded target polynucleotide of unknown length A to a single-stranded probe polynucleotide of predetermined fixed length B; wherein said length A is shorter than said length B;
detecting a number of target polynucleotides that are hybridized to said probe polynucleotide; and
determining said length A based on said detecting step.

29. The method of claim 28, wherein
said probe polynucleotide and said target polynucleotide are deoxyribonucleic acid (DNA).

30. The method of claim 28, wherein
said probe polynucleotide and said target polynucleotide respectively includes a finite number of short tandem repeat (STR) sequences; and wherein
said length A and said length B are respectively determined by said number of STR sequences contained in said probe polynucleotide and said target polynucleotide, respectively.

31. The method of claim 30, wherein
said probe polynucleotide contains about twice or more STR sequences than said target polynucleotide.

32. The method of claim 28, further comprising
attaching at least one polynucleotide probe to a predetermined position on surface of a microarray.

33. The method of claim 32, further comprising
modifying said polynucleotide probe on its 5′ or 3′ end with a chemical entity to allow said end to attach covalently or noncovalently to said microarray surface.

34. The method of claim 32, further comprising
utilizing a chemical or biological linker to attach said probe polynucleotide to said microarray.

35. The method of claim 32, wherein
said probe polynucleotide contains a spacer sequence; and wherein
said spacer sequence allows a repeat sequence to protrude into a solution and away from said surface of said microarray.

36. The method of claim 28, further comprising
modifying said probe polynucleotide with a sulfur-containing group; and
attaching said modified probe polynucleotide through a sulfur linkage to a substrate.

37. The method of claim 28, further comprising
labeling said target polynucleotide on its 5′ or 3′ end with a fluorescent dye, a superparamagnetic particle, or a synthetic antiferromagnetic particle.

38. The method of claim 37, wherein
said fluorescent dye is Cy3 or Cy5.

39. The method of claim 28, further comprising
attaching said target polynucleotide to an end-label with a chemical means, a biological means, or a physical linker.

40. The method of claim 28, further comprising
labeling said probe polynucleotide, said target polynucleotide, or both, with at least one molecule at a position that is neither 5′ end nor 3′ end.

41. The method of claim 28, further comprising
employing fluorescence detection or magnetic detection to detect said number of target polynucleotides that are hybridized to said probe polynucleotide.

42. The method of claim 28, wherein
said probe polynucleotide has a surface concentration at hybridization sites that is substantially smaller than that of said target polynucleotide.

43. The method of claim 28, further comprising
deducing said number of target polynucleotides by
gradually denaturing hybrids such that shorter hybrids denature at lower temperatures than longer hybrids; and
detecting said denaturation in real time.

44. A method for single nucleotide polymorphism (SNP) detection comprising attaching at least one polynucleotide probe to surface of a microarray;
hybridizing at least one labeled single-stranded polynucleotide target to said probe to form target-probe hybrids;
denaturing said hybrids; and
monitoring said denaturation in real time as labeled targets are removed from said microarray.

45. The method of claim 44, wherein
sequences of said probe and said target are either fully complimentary or contain a single-base mismatch.

46. The method of claim 45, further comprising
determining which hybrids exhibit a decrease in signal upon denaturation.

47. The method of claim 44, further comprising
applying heat or chemicals to denature said hybrids.

48. The method of claim 44, further comprising
modifying said probe on its 5′ or 3′ end with a chemical entity to allow said end to attach covalently or noncovalently to said microarray.

49. The method of claim 44, further comprising
utilizing a chemical or biological linker to attach said probe to said microarray.

50. The method of claim 44, further comprising
modifying said probe with a sulfur-containing group; and
attaching said modified probe through a sulfur linkage to a substrate.

51. The method of claim 44, further comprising
modifying said probe on its 5′ or 3′ end with an amine group.

52. The method of claim 44, further comprising
labeling said target on its 5′ or 3′ end with a fluorescent dye, a superparamagnetic particle, or a synthetic antiferromagnetic particle.

53. The method of claim 52, wherein
said fluorescent dye is Cy3 or Cy5.

54. The method of claim 44, further comprising
attaching said target to an end-label with a chemical means, a biological means, or a physical linker.

55. The method of claim 44, further comprising
labeling said probe polynucleotide, said target polynucleotide, or both, with at least one molecule at a position that is neither 5′ end nor 3′ end.

56. The method of claim 44, further comprising
employing fluorescence detection or magnetic detection during said monitoring step.
